(19) United States
(12) Patent Application Publication
(10) Pub. No.: US 2023/0397953 A1
(43) Pub. Date: Dec. 14, 2023

(54) AUTOMATED LASER ABLATION CONTROLLER, SYSTEM, AND METHODS

(71) Applicant: Medtronic Navigation, Inc., Louisville, CO (US)

(72) Inventors: Christine Drown, Westminster, CO (US); Rebecca Vincelette, Arvada, CO (US); Matthew W. Koenig, Denver, CO (US)

(21) Appl. No.: 18/206,513

(22) Filed: Jun. 6, 2023

Related U.S. Application Data
(60) Provisional application No. 63/366,136, filed on Jun. 9, 2022.

(51) Int. Cl. A61B 18/20; A61B 90/00

(52) U.S. Cl. CPC A61B 18/201; A61B 90/37; A61B 2018/00577

(57) ABSTRACT

Systems and methods for automated operation of laser ablation system for disrupting target tissue via heat application are disclosed. The system includes a controller coupled to various devices, such as a magnetic resonance imaging device, a laser energy source, a laser fiber manipulating device, a laser fiber cooling device, and a tissue damage analysis computer system via a communication interface. The controller automatically controls various functions of the coupled devices during a laser ablation procedure while monitoring tissue temperature at a laser ablation site.

### CROSS-REFERENCE TO RELATED APPLICATIONS

The present application claims the benefit of and priority, under 35 U.S.C. § 119(e), to U.S. Provisional patent application Ser. No. 63/366,136, filed on Jun. 9, 2022, entitled “AUTOMATED LASER ABLATION CONTROLLER, SYSTEM, AND METHODS,” the entire disclosure of which is hereby incorporated herein by reference, in its entirety, for all that it teaches and for all purposes.

### BACKGROUND

The present disclosure relates generally to the field of medical devices. More particularly, the present disclosure relates to automated tissue ablation controllers, systems, and methods.

Tissue ablation procedures are capable of utilizing a focused energy to necrotize, remove, or otherwise destroy an area of target tissue associated with a patient. The focused energy is typically provided in the form of laser energy, radio frequency energy, ultrasound energy, and/or some other form of thermal energy.

### BRIEF SUMMARY

Example aspects of the present disclosure include:

A thermal ablation control system according to at least one embodiment of the present disclosure comprises: a memory to store user input values defining elements of a thermal ablation procedure; and a controller comprising: a communication interface coupled to a thermal energy source, a thermal fiber positioning device, and a magnetic resonance imaging (MRI) system; and a processor to: monitor a magnetic resonance (MR) thermometry tissue temperature at an ablation site; and automatically control functions of the thermal energy source, the thermal fiber positioning device, and the MRI system via the communication interface during thermal ablation of a target tissue based on the MR thermometry tissue temperature at the ablation site and the stored user input values.

Any of the aspects herein, wherein the thermal energy source is configured to output a laser energy having a wavelength, a power level, and a pulse frequency.

Any of the aspects herein, wherein the controller is configured to automatically control the wavelength, power level, and pulse frequency of the thermal energy source.

Any of the aspects herein, wherein the thermal fiber positioning device comprises: an axial positioning mechanism; and a directionality mechanism.

Any of the aspects herein, wherein the controller is configured to automatically control the axial positioning mechanism and the directionality mechanism.

Any of the aspects herein, wherein the MRI system comprises an MR thermometry function configured to non-invasively estimate a tissue temperature and a thermal fiber locating function configured to orient the MM system to locate a thermal fiber relative to the ablation site.

Any of the aspects herein, wherein the controller is configured to automatically control the MR thermometry function and the thermal fiber locating function.

Any of the aspects herein, wherein the communication interface is further coupled to a thermal fiber cooling system; and wherein the controller is configured to automatically control functions of the thermal fiber cooling system via the communication interface during thermal ablation of the target tissue.

Any of the aspects herein, wherein the thermal fiber cooling system comprises a fluid pump.

Any of the aspects herein, wherein the controller is configured to control a flowrate of the fluid pump.

Any of the aspects herein, wherein the controller is configured to receive the stored user input values.

Any of the aspects herein, wherein the user input values comprise: a protected tissue area; and a target tissue area.

Any of the aspects herein, wherein the controller is configured to receive a pre-calculated tissue damage model.

A method of controlling a thermal ablation system according to at least one embodiment of the present disclosure comprises: receiving user input values at a controller coupled to a thermal energy source, a thermal fiber positioning device, and a magnetic resonance imaging (MRI) system; activating the thermal ablation system via the controller; monitoring a magnetic resonance (MR) thermometry tissue temperature at an ablation site; and automatically controlling, via the controller, the thermal energy source, the thermal fiber positioning device, and the MRI system during a thermal tissue ablation procedure based on the MR thermometry tissue temperature at the ablation site.

Any of the aspects herein, wherein the user input values are input via a graphical user interface (GUI). Any of the aspects herein, wherein automatically controlling the thermal energy source comprises controlling a wavelength, a power level, and a pulse frequency of a laser energy output of the thermal energy source.

Any of the aspects herein, wherein automatically controlling the thermal fiber positioning device comprises: controlling an axial positioning mechanism; and controlling a directionality mechanism.

Any of the aspects herein, wherein automatically controlling the MM system comprises controlling an MR thermometry function configured to non-invasively estimate a tissue temperature and a thermal fiber location function configured to orient the MM system to locate a thermal fiber relative to the ablation site.

Any of the aspects herein, further comprising automatically controlling a thermal fiber cooling device comprising a fluid pump.

Any of the aspects herein, wherein automatically controlling functions of the thermal fiber cooling device comprises controlling a flowrate of the fluid pump.

Any of the aspects herein, further comprising receiving of a pre-calculated tissue damage model by the controller.

A thermal ablation system according to at least one embodiment of the present disclosure comprises: a thermal energy source; a thermal fiber positioning device; a magnetic resonance imaging (MRI) system; a thermal fiber cooling device; and a controller comprising a communication interface coupled to the thermal energy source, the thermal fiber positioning device, the MM system, and the thermal fiber cooling system; wherein the controller is configured to automatically control functions of the thermal energy source, the thermal fiber positioning device, the MRI system, and the thermal fiber cooling device via the communication interface during thermal ablation of a target tissue.

Any of the aspects herein, wherein the thermal energy source is configured to output a laser energy having a wavelength, a power level, and a pulse frequency.

Any of the aspects herein, wherein the controller is configured to automatically control the wavelength, power level, and pulse frequency of the laser energy output of the thermal energy source.

Any of the aspects herein, wherein the thermal fiber positioning device comprises: an axial positioning mechanism; and a directionality mechanism.

Any of the aspects herein, wherein the controller is configured to automatically control the axial positioning mechanism and the directionality mechanism.

Any of the aspects herein, wherein the MRI system comprises a magnetic resonance (MR) thermometry function and a thermal fiber locating function.

Any of the aspects herein, wherein the controller is configured to automatically control the MR thermometry function and the thermal fiber locating function.

Any of the aspects herein, wherein the thermal fiber cooling device comprises a fluid pump.

Any of the aspects herein, wherein the controller is configured to control a flowrate of the fluid pump.

Any of the aspects herein, wherein the controller is configured to receive user input values.

Any of the aspects herein, wherein the user input values comprise: a protected tissue area; and a target tissue area.

Any of the aspects herein, wherein the controller is configured to receive a pre-calculated tissue damage model.

Any aspect in combination with any one or more other aspects.

Any one or more of the features disclosed herein.

Any one or more of the features as substantially disclosed herein.

Any one or more of the features as substantially disclosed herein in combination with any one or more other features as substantially disclosed herein.

Any one of the aspects/features/embodiments in combination with any one or more other aspects/features/embodiments.

Use of any one or more of the aspects or features as disclosed herein.

It is to be appreciated that any feature described herein can be claimed in combination with any other feature(s) as described herein, regardless of whether the features come from the same described embodiment.

The details of one or more aspects of the disclosure are set forth in the accompanying drawings and the description below. Other features, objects, and advantages of the techniques described in this disclosure will be apparent from the description and drawings, and from the claims.

The phrases “at least one,” “one or more,” “or,” and “and/or” are open-ended expressions that are both conjunctive and disjunctive in operation. For example, each of the expressions “at least one of A, B and C,” “at least one of A, B, or C,” “one or more of A, B, and C,” “one or more of A, B, or C,” and “A, B, and/or C” means A alone, B alone, C alone, A and B together, A and C together, B and C together, or A, B, and C together. When each one of A, B, and C in the above expressions refers to an element, such as X, Y, and Z, or a class of elements, such as X1-Xn, Y1-Ym, and Z1-Zo, the phrase is intended to refer to a single element selected from X, Y, and Z, a combination of elements selected from the same class (e.g., X1 and X2) as well as a combination of elements selected from two or more classes (e.g., Y1 and Zo).

The term “a” or “an” entity refers to one or more of that entity. As such, the terms “a” (or “an”), “one or more,” and “at least one” can be used interchangeably herein. It is also to be noted that the terms “comprising,” “including,” and “having” can be used interchangeably.

The preceding is a simplified summary of the disclosure to provide an understanding of some aspects of the disclosure. This summary is neither an extensive nor exhaustive overview of the disclosure and its various aspects, embodiments, and configurations. It is intended neither to identify key or critical elements of the disclosure nor to delineate the scope of the disclosure but to present selected concepts of the disclosure in a simplified form as an introduction to the more detailed description presented below. As will be appreciated, other aspects, embodiments, and configurations of the disclosure are possible utilizing, alone or in combination, one or more of the features set forth above or described in detail below.

Numerous additional features and advantages are described herein and will be apparent to those skilled in the art upon consideration of the following Detailed Description and in view of the figures.

### BRIEF DESCRIPTION OF THE SEVERAL VIEWS OF THE DRAWINGS

The accompanying drawings are incorporated into and form a part of the specification to illustrate several examples of the present disclosure. These drawings, together with the description, explain the principles of the disclosure. The drawings simply illustrate preferred and alternative examples of how the disclosure can be made and used and are not to be construed as limiting the disclosure to only the illustrated and described examples. Further features and advantages will become apparent from the following, more detailed, description of the various aspects, embodiments, and configurations of the disclosure, as illustrated by the drawings referenced below.

FIG. 1 illustrates a block diagram of at least one embodiment of an automated laser ablation system;

FIG. 2 illustrates a block diagram of at least one embodiment of a controller of the automated laser ablation system of FIG. 1;

FIG. 3 illustrates a block diagram of at least one embodiment of a tissue damage analysis computer system of the automated laser ablation system of FIG. 1;

FIG. 4 illustrates the automated laser ablation system of FIG. 1; and

FIG. 5 illustrates a flowchart of a method, according to at least one embodiment of the present disclosure, for performing an automated laser tissue ablation.

### DETAILED DESCRIPTION

It should be understood that various aspects disclosed herein may be combined in different combinations than the combinations specifically presented in the description and accompanying drawings. It should also be understood that, depending on the example or embodiment, certain acts or events of any of the processes or methods described herein may be performed in a different sequence, and/or may be added, merged, or left out altogether (e.g., all described acts or events may not be necessary to carry out the disclosed techniques according to different embodiments of the present disclosure). In addition, while certain aspects of this disclosure are described as being performed by a single module or unit for purposes of clarity, it should be understood that the techniques of this disclosure may be performed by a combination of units or modules associated with, for example, a computing device and/or a medical device.

In one or more examples, the described methods, processes, and techniques may be implemented in hardware, software, firmware, or any combination thereof. If implemented in software, the functions may be stored as one or more instructions or code on a computer-readable medium and executed by a hardware-based processing unit. Alternatively or additionally, functions may be implemented using machine learning models, neural networks, artificial neural networks, or combinations thereof (alone or in combination with instructions). Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors (e.g., Intel Core i3, i5, i7, or i9 processors; Intel Celeron processors; Intel Xeon processors; Intel Pentium processors; AMD Ryzen processors; AMD Athlon processors; AMD Phenom processors; Apple A10 or 10X Fusion processors; Apple A11, A12, A12X, A12Z, or A13 Bionic processors; or any other general purpose microprocessors), graphics processing units (e.g., Nvidia GeForce RTX 2000-series processors, Nvidia GeForce RTX 3000-series processors, AMD Radeon RX 5000-series processors, AMD Radeon RX 6000-series processors, or any other graphics processing units), application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

Before any embodiments of the disclosure are explained in detail, it is to be understood that the disclosure is not limited in its application to the details of construction and the arrangement of components set forth in the following description or illustrated in the drawings. The disclosure is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items. Further, the present disclosure may use examples to illustrate one or more aspects thereof. Unless explicitly stated otherwise, the use or listing of one or more examples (which may be denoted by “for example,” “by way of example,” “e.g.,” “such as,” or similar language) is not intended to and does not limit the scope of the present disclosure.

The terms proximal and distal are used in this disclosure with their conventional medical meanings, proximal being closer to the operator or user of the system, and further from the region of surgical interest in or on the patient, and distal being closer to the region of surgical interest in or on the patient, and further from the operator or user of the system.

The ensuing description provides embodiments only, and is not intended to limit the scope, applicability, or configuration of the claims. Rather, the ensuing description will provide those skilled in the art with an enabling description for implementing the described embodiments. It being understood that various changes may be made in the function and arrangement of elements without departing from the spirit and scope of the appended claims.

Various aspects of the present disclosure will be described herein with reference to drawings that may be schematic illustrations of idealized configurations.

During ablation procedures, damage to tissue is dependent on both temperature and time. For example, while tissue will necrotize more quickly when higher temperatures are applied, even lower temperatures can damage tissue when applied for relatively long periods of time. As such, tissue near target tissue can also be unintentionally damaged by either high temperatures or by lower temperature heat that is applied for long periods of time. Such issues can be particularly problematic when critical tissue is located near the tissue to be disrupted (e.g., the brain stem near a tumor to be necrotized, etc.).

In conventional laser ablation products, the system provides a user with information to guide their decision making in how to progress the treatment (e.g., laser power, duration). The user must have advanced understanding of the thermodynamic environment, and independently control the laser power, wavelength, duration, flow, etc. This requires experience with laser ablation, which limits the ability to broadly and safely scale up product offerings without significant training and clinical specialist support.

It is with respect to the above issues and other problems that the embodiments presented herein were contemplated. For example, at least some embodiments of the system described herein may have (e.g., through user input or otherwise) information about surgical targets to be destroyed by the ablation, as well as nearby structures to be spared from laser ablation. The system may also have access to MM thermometry and damage models (e.g., pre-calculated tissue damage models, etc.). The system can use the information to automatically control the laser and any repositioning device (e.g., such as a pullback device, etc.) to optimally damage the surgical target while protecting nearby critical structures. Aspects of the device that can control include, but are in no way limited to, laser wavelength, laser power, laser duration, number of laser on events, cooling flow, positioning/location of laser emission and/or the like.

In contrast to conventional ablation methods and systems that rely on a user to control the laser and cooling systems within a laser ablation product, at least one embodiment of the system of the present disclosure can automatically control the system in a feedback loop with incoming MM thermometry data (e.g., without requiring user input and/or control). In some embodiments, the system of the present disclosure is capable of optimizing the spread of heat in the brain to maximize the ablation of targeted tissue, while minimizing the collateral damage to nearby structures. As ablation solutions become more complicated (e.g., requiring additional wavelengths, pullback devices, etc.) this optimization can greatly simplify that number of parameters that a user must control and understand to have a safe and effective treatment.

Tissue ablation devices can be used to treat problematic tissue within the body of a patient (e.g., a tumor on the brain of a patient). For instance, as an initial step of a tissue ablation procedure, a surgeon may use an image of tissue (e.g., captured using Magnetic Resonance Imaging (MRI)) that includes the area to be treated (also referred to herein as target tissue) in conjunction with computer software to identify the target tissue and create a surgical plan that allows for treating the target tissue while attempting to avoid any healthy surrounding tissue or critical structures (e.g., the patient's brain stem). In the example of treating a patient's brain tissue, a small entry (e.g., between 3 and 5 millimeters) can be made in the skull of the patient. An MRI-compatible bone anchor may then be fixed to the entry to allow for the introduction and securing of a laser applicator.

The laser applicator can then be inserted into the target tissue. The laser applicator may include a laser fiber that is then connected to a laser energy source. The patient is placed in the MRI and imaged in preparation for the destruction of the target. The operator (e.g., a physician) can then review new images captured using the MM to verify that the laser applicator is positioned properly within the target tissue. A low-power test pulse can be delivered as a second confirmation of precise placement of the laser applicator. The laser power is then increased, causing light energy to be absorbed by the target tissue, thus heating and disrupting the target tissue.

While the example above relates specifically to performing such ablation with respect to the brain, ablation procedures associated with the prostate, kidney, liver, cardiovascular thoracic surgery, dermatology, ear-nose-throat surgery, gastroenterology, general surgery, gynecology, head and neck surgery, neurosurgery, plastic surgery, orthopedics, pulmonology, radiology, and urology may be similarly applicable when practicing the principles described herein. Similarly, the principles described herein may be equally applicable to any procedure where the temperature of tissue is to be monitored (e.g., laser ablation, High-Intensity Focused Ultrasound (HIFU), and so forth). Regardless of the location/type of such tissue or the exact type of procedure, when disrupting problematic tissue using heat, a number of issues can arise. Notably, tissue damage is dependent on both temperature and time. Accordingly, while tissue will necrotize more quickly when higher temperatures are applied, even lower temperatures can damage tissue when applied for relatively long periods of time. As such, tissue near the problematic tissue that is being purposely destroyed can also be unintentionally damaged by either high temperatures or lower temperature heat applied for long periods of time. Such issues can be particularly problematic when critical tissue is located near the tissue to be disrupted (e.g., the brain stem near a tumor to be necrotized).

Accordingly, disclosed within the scope of this disclosure is an embodiment of an automated laser ablation system for automatically necrotizing target tissue and protecting non-target tissue from damage while monitoring a magnetic resonance (MR) thermometry tissue temperature during a medical procedure for disrupting tissue via heat application (e.g., laser ablation procedures, HIFU procedures, and so forth). As disclosed, the automated ablation system may include various devices, computer systems, engines, and components, including a controller (e.g., an omnibus controller, universal controller, etc.), a graphical user interface (GUI), a communication interface, a memory, a processor, an MM device, a thermal energy source (e.g., a laser energy source, etc.), a thermal fiber positioning device (e.g., a laser fiber positioning device, etc.), a thermal fiber cooling device (e.g., a laser fiber cooling device, etc.), and a tissue damage analysis computer system. In other embodiments, the automated laser ablation system may include more or fewer than the devices, computer systems, and/or components illustrated previously mentioned. The various devices, computer systems, engines, and/or components of the automated ablation system may include functionality that is implemented as software, hardware, or a combination of software and hardware.

In use, user input values are inputted by a user using the GUI and stored in the memory. The controller receives the stored user input values and automatically activates the automated laser ablation system to initiate a laser ablation procedure. During the automated laser ablation procedure, the processor of the controller communicates data and instructions via the communications interface with one or more of the MRI device, the laser energy source, the laser fiber positioning device, the laser fiber cooling device, and the tissue damage analysis computer system. The controller may automatically control various functions of the MRI device, the laser energy source, the laser fiber positioning device, the laser fiber cooling device, and the tissue damage analysis computer system as it monitors the MR thermometry tissue temperature at the ablation site. The automated laser ablation procedure can necrotize target tissue and protect non-target tissue from damage.

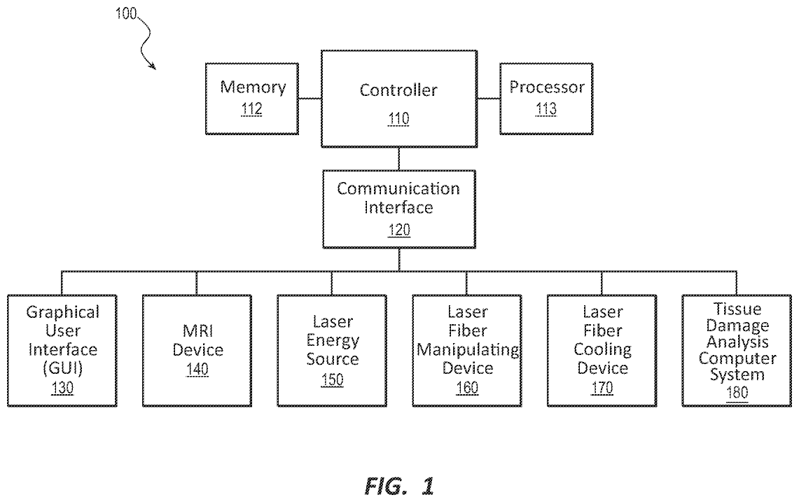

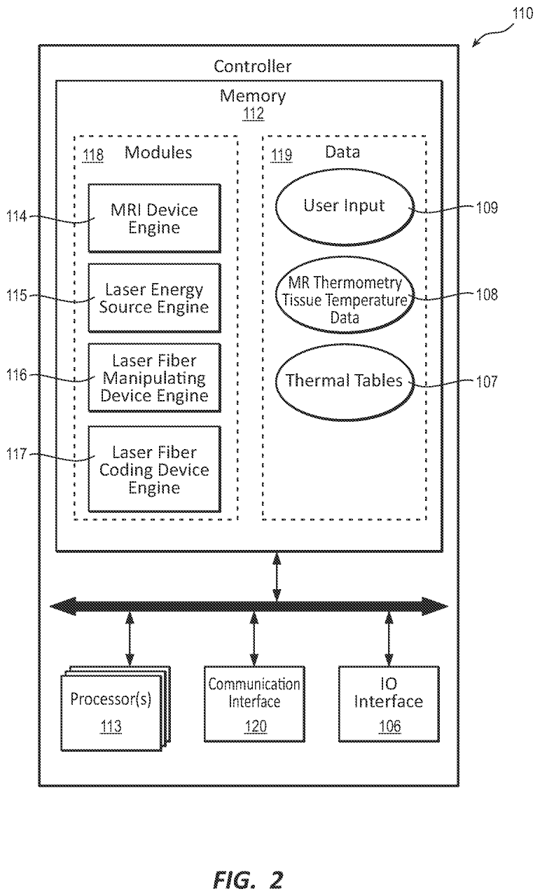

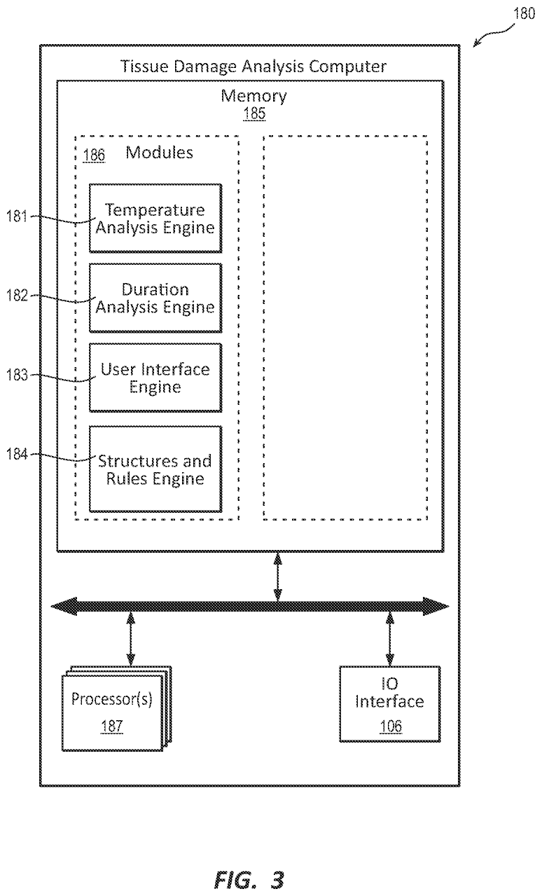

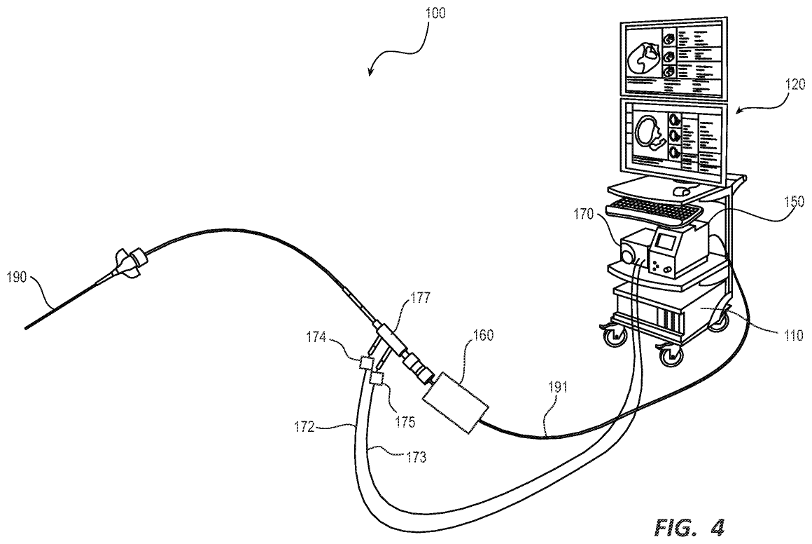

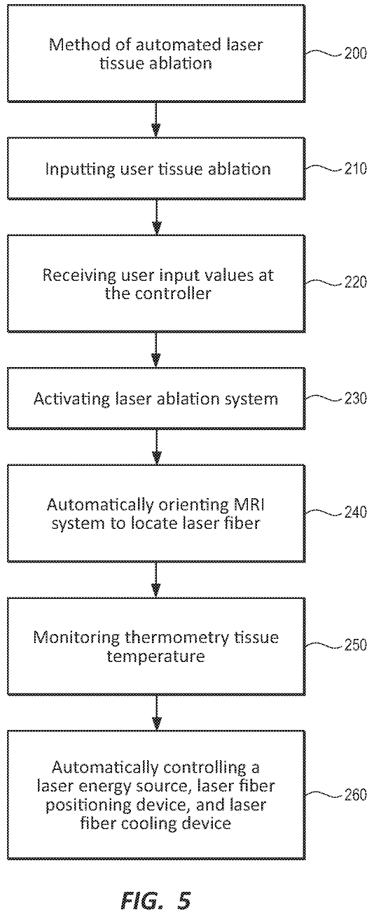

FIG. 1 illustrates a block diagram of at least one embodiment of an automated laser ablation system. FIG. 2 illustrates a block diagram of at least one embodiment of a controller of the automated laser ablation system. FIG. 3 illustrates a block diagram of at least one embodiment of a tissue damage analysis computer system. FIG. 4 illustrates the automated laser ablation system. FIG. 5 illustrates a flowchart of a method, according to at least one embodiment of the present disclosure, for performing an automated laser tissue ablation. In certain views each device may be coupled to, or shown with, additional components not included in every view. Further, in some views only selected components are illustrated, to provide detail into the relationship of the components. Some components may be shown in multiple views, but not discussed in connection with every view. Disclosure provided in connection with any figure is relevant and applicable to disclosure provided in connection with any other figure, example, or embodiment.

FIG. 1 illustrates at least one embodiment of an automated laser ablation system 100 for automatically necrotizing target tissue and protecting non-target tissue from damage while monitoring an MR thermometry tissue temperature during a medical procedure for disrupting tissue via heat application (e.g., laser ablation procedures, HIFU procedures, and so forth). As disclosed herein, the automated laser ablation system 100 may include various devices, computer systems, engines, and components, including a controller (e.g., an omnibus controller, universal controller, etc.) 110, a memory 112, a processor 113, a communication interface 120, a graphical user interface (GUI) 130, an MRI device 140, a laser energy source 150, a laser fiber manipulating device 160, a laser fiber cooling device 170, and a tissue damage analysis computer system 180. In other embodiments, the automated laser ablation system 100 may include more than, or a subset of, the devices, computer systems, and/or components illustrated in FIG. 1. The various devices, computer systems, engines, and/or components of the automated laser ablation system 100 may include functionality that is implemented as software, hardware, or a combination of software and hardware.

The controller 110 is illustrated with more specificity in FIG. 2. As shown, the controller can include the memory 112, the processor 113, modules 118, the communication interface 120, and an I/O interface 106. The controller 110 may be configured to automatically control some functions of the MRI device 140, the laser energy source 150, the laser fiber manipulating device 160, and the laser fiber cooling device 170 via the modules 118 after receiving physician input through the GUI 130 and while monitoring tissue temperature at the ablation site.

The processor 113 may be used to process executable code and data stored in the memory 112. The memory 112 may include static RAM, dynamic RAM, flash memory, one or more flip-flops, or other electronic (e.g., computer readable) storage medium. The electronic memory 112 may include a plurality of engines or modules 118 and data 119. The modules 118 may run multiple operations serially, concurrently or in parallel on the one or more processors 113.

In some embodiments, portions of the disclosed engines, components, and/or facilities are embodied as executable instructions embodied in hardware or in firmware, or stored on a non-transitory, machine-readable storage medium. The instructions may comprise computer program code that, when executed by a processor and/or computing device, cause a computing system to implement certain processing steps, procedures, and/or operations, as disclosed herein. The modules, components, and/or facilities disclosed herein may be implemented and/or embodied as a driver, a library, an interface, an API, FPGA configuration data, firmware (e.g., stored on an EEPROM), and/or the like. In some embodiments, portions of the modules, components, and/or facilities disclosed herein are embodied as machine components, such as general and/or application-specific devices, including, but not limited to: circuits, integrated circuits, processing components, interface components, hardware controller(s), storage controller(s), programmable hardware, FPGAs, ASICs, and/or the like. A software module or component may include any type of computer instruction or computer executable code located within or on a computer-readable storage medium. A software module may, for instance, comprise one or more physical or logical blocks of computer instructions, which may be organized as a routine, program, object, component, data structure, etc., that performs one or more tasks or implement particular abstract data types. A particular software module may comprise disparate instructions stored in different locations of a computer-readable storage medium, which together implement the described functionality of the module. Indeed, a module may comprise a single instruction or many instructions, and may be distributed over several different code segments, among different programs, and across several computer-readable storage media.

In one embodiment, the modules 118 include an MRI device engine 114, a laser energy source engine 115, a laser fiber manipulating device engine 116, and a laser fiber cooling device engine 117. In other embodiments, additional and/or alternative modules 118 are contemplated. In certain embodiments, the data 119 may include user input 109, MR thermometry tissue temperature data 108, and thermal tables 107. In other embodiments, additional and/or alternative data 119 are contemplated. The communication interface 120 can be configured to provide a communication protocol allowing the controller 110 to communicate (e.g., send and receive data, instructions, etc.) with each of the GUI 130, the MRI device 140, the laser energy source 150, the laser fiber manipulating device 160, the laser fiber cooling device 170, and the tissue damage analysis computer system 180 over a network. The communication interface 120 can be of any suitable type configured to communicate digital data between the various components of the automated laser ablation system 100, such as wired or wireless network connectivity technologies.

The MRI device 140 of the illustrated embodiment of FIG. 1 may comprise any suitable type of MRI device that is capable of generating images using MRI technology and providing MR thermometry tissue temperature data to the controller 110. The MR thermometry tissue temperature data may be obtained by using proton resonance frequency (PRF) MR thermometry where a decreasing resonance frequency of water protons decreases with increasing temperature or any other suitable method. The MRI device 140 may comprise a closed MRI machine, an open MM machine, a 3 Tesla MRI machine, and so forth.

The laser energy source 150 of the illustrated embodiment of FIG. 1 may comprise any suitable type of laser energy generator. In certain embodiments, the laser energy source 150 can be adjusted to vary the power or intensity level of the laser energy ranging from about 1 W to about 40 W. In some embodiments, the laser energy source 150 may generate and transmit laser energy having a single wavelength ranging between about 800 nm and about 1310 nm and may be about 980 nm. In other embodiments, the laser energy source 150 can generate and transmit laser energy having two or more wavelengths to optimize necrotization of the target tissue. For example, in certain embodiments, the laser energy source 150 may generate and transmit laser energy comprising wavelengths of 980 nm and 1064 nm. In other embodiments, the laser energy source may transmit laser energy comprising wavelengths of 800 nm, 980 nm, and 1064 nm. The laser energy source 150 can transmit the laser energy as a continuous or a pulsed output with a frequency ranging from about 1 cycle/second to about 150 cycles/second and from about 5 cycles/second to about 97 cycles/second.

The laser fiber manipulating device 160 of the illustrated embodiment of FIG. 1 may comprise an axial positioning mechanism to axially translate a laser fiber proximally and distally during an ablation procedure. The axial translation of the laser fiber can change the position of a distal end of the laser fiber relative to the target tissue to facilitate dispersion of laser energy from the distal end along a length of the target tissue. In some embodiments, the axial translation is a continuous motion. In other embodiments, the axial translation is a stepwise movement. The laser fiber manipulating device 160 can also include a directionality mechanism to direct the distal end of the laser fiber in a desired direction. The directionality mechanism may bend the distal end in an arcuate shape to direct the distal end radially outward from the longitudinal axis of the laser fiber. The bending of the distal end can range from 0 degrees to about 60 degrees (plus or minus 5 to 25 degrees), in any direction, measured from the longitudinal axis of the laser fiber. In some embodiments, the directionality mechanism may also rotate the laser fiber about a longitudinal axis. The rotation may be from about one degree to about 360 degrees.

As illustrated in FIG. 4, the automated laser ablation system 100 may include the laser energy source 150, the laser fiber manipulating device 160, the laser fiber cooling device 170, a laser applicator 190, and a laser fiber 191 operably coupled together. The laser applicator 190 may include a closed-end catheter that is configured to be placed within or adjacent a tissue (e.g., target tissue) to be damaged/necrotized and facilitate the provision of laser energy that is absorbed by such tissue. A distal portion of the laser fiber 191 may be disposed within the laser applicator 190 and a proximal end, or portion, thereof may be connected to the laser energy source 150. Accordingly, the laser energy source 150 may provide laser energy that is transmitted via the laser fiber 191 and the laser applicator 190 to the target tissue to be disrupted.

As shown in FIG. 4, the laser fiber 191 can be operatively coupled to the laser fiber manipulating device 160. In certain embodiments, the laser fiber manipulating device 160 may proximally displace or pull back the laser fiber 191 relative to the laser applicator 190. Such displacement can occur during an ablation procedure to position a distal end of the laser fiber 191 at various locations over the length of the laser applicator 190, resulting in laser energy directed to various locations along the length of the target tissue. In other embodiments, the laser fiber manipulating device 160 may manipulate the distal end of the laser fiber 191 to provide radial directionality to the laser energy dispersed from the distal end.

As illustrated in FIG. 4, the laser fiber cooling device 170 of the automated laser ablation system 100 may be coupled to the laser fiber 191 and the laser applicator 190. The laser fiber cooling device 170 may be configured to circulate fluid (e.g., saline, etc.) around the laser fiber 191 within the laser applicator 190 to draw heat from the laser fiber 191 to maintain a workable temperature of the laser fiber 191 during the ablation procedure. The laser fiber cooling device 170 may include a fluid pump 171 in fluid communication with a fluid reservoir 176 and a manifold 177 through inflow tubing 172 and outflow tubing 173. The manifold 177 can be coupled to the laser applicator 190, and the laser fiber 191 can pass through the manifold 177. The manifold 177 may include an inflow port 174 coupled to the inflow tubing 172 and an outflow port 175 coupled to the outflow tubing 173. An inflow sensor may be coupled to, or otherwise associated with, the inflow tubing 172 and/or inflow port 174, and an outflow sensor may be coupled to, or otherwise associated with, the outflow tubing 173 and/or outflow port 175. These sensors (e.g., at the inflow port 174 and the outflow port 175, etc.) may measure fluid temperature and/or fluid flowrate to determine proper function of the laser fiber cooling device 170 and/or the automated laser ablation system 100.

As illustrated in FIG. 4, the proximal end of the laser fiber 191 may be coupled to the laser energy source 150. The laser energy source 150 can generate laser energy and transmit the laser energy to the laser fiber 191 for transmission to the distal end of the laser fiber 191 and dispersion into the target tissue.

While laser ablation is discussed frequently herein, the principles discussed throughout this disclosure may be equally applicable to any procedure where the temperature of tissue is to be monitored. For instance, HIFU procedures may similarly benefit from the principles described herein. Regardless of the location/type of such tissue or the exact type of procedure, when disrupting problematic tissue using heat, a number of issues can arise. Notably, tissue damage is dependent on both temperature and time. Accordingly, while tissue will necrotize more quickly when higher temperatures are applied, even lower temperatures can damage tissue when applied for relatively long periods of time. As such, tissue near target tissue can also be unintentionally damaged by either high temperatures or lower temperature heat applied for long periods of time. Such issues can be particularly problematic when critical tissue is located near the tissue to be disrupted (e.g., the brain stem near a tumor to be necrotized).

As illustrated in FIG. 1, the automated laser ablation system 100 includes the tissue damage analysis computer system 180, which is configured to analyze both the temperature and duration of applied temperature associated with multiple locations of tissue affected by automated laser ablation system 100. In addition, the tissue damage analysis computer system 180 may be configured to assist the automated laser ablation system 100 in avoiding unintentionally disrupting tissue located adjacent to or near target tissue (e.g., by automatically shutting off power to the ablation device, by lowering the temperature applied to target tissue, by warning the end user of potential unintentional damage, and so forth), as further described herein.

As shown in FIG. 3, the tissue damage analysis computer system 180 may include various engines or modules 186 stored on a memory 185 and direct a processor 187 to perform actions. The engines may include a temperature analysis engine 181, a duration analysis engine 182, a user interface (UI) engine 183, and a structures and rules engine 184. In other embodiments, the additional and/or alternative engines are contemplated. In certain embodiments, the UI engine 183 may control a user interface on an I/O interface 106, such as a display. In some embodiments, the tissue damage analysis computer system 180 may be incorporated with the controller 110.

The temperature analysis engine 181 may be configured to measure and monitor the temperature of various objects of tissue in addition to the target tissue in real-time during an ablation procedure. Such measurements and monitoring may be performed by utilizing various technologies. In an example, the temperature analysis engine 181 may utilize MR thermometry. In another example, a probe (e.g., the laser applicator 190) used during a procedure may include one or more physical temperature sensors that are configured to monitor the temperature of nearby or adjacent tissue.

Notably, the objects of tissue being monitored by the temperature analysis engine 181 may be any definable object or structure of tissue. For instance, the temperature analysis engine 181 may monitor the temperature at various zones or voxels correlated to tissue. In addition, such objects may include user-defined objects that are defined through software, computer-defined objects that are defined through machine learning, tissue structures identified by the tissue damage analysis computer system based on predefined objects (e.g., via the structures and rules engine 184), and so forth. In a specific example, such objects may comprise brain fiber tracts viewable through diffusion tensor imaging. In another example, a model of an object or structure may be built by a user by hand or created by a computer system (e.g., via the structures and rules engine 184, as further described herein). In a more particular example, an end user (e.g., a physician) may create an object of tissue to be monitored for temperature using a surgical planning software via the UI engine 183.

As briefly described above, the tissue damage analysis computer system 180 also includes the duration analysis engine 182. The duration analysis engine 182 may be configured to determine the duration (e.g., an amount of time, etc.) at which any given tissue (e.g., structures, objects, voxels, zones, and so forth, as further described herein) has been placed at an elevated temperature. In other words, the duration analysis engine 182 may determine the thermal dose (the accumulated thermal energy) that has been applied to any given tissue during a procedure. In some embodiments, the duration analysis engine 182 may utilize one or more models that assist in making such determinations. For instance, the Arrhenius model may be used to assist in determining the duration at which any given tissue has been placed at an elevated temperature. In another example, the cumulative equivalent minutes at 43 degrees C. (CEM43) model may be used to assist in making such determinations. While two examples of particular models are discussed, any model (e.g., tissue damage model, etc.) that assists in estimating both temperature and duration (e.g., amount of time at an elevated temperature) may be used.

As illustrated in FIG. 3, the tissue damage analysis computer system 180 also includes the UI engine 183. The UI engine 183 may be configured to receive various types of input from an end user. For instance, the UI engine 183 may receive input from an end user regarding the particular tissue objects to be monitored. In another example, the UI engine 183 may also allow an end user to identify and create particular objects of tissue (e.g., voxels, zones, and so forth) to be monitored. In yet another example, the UI engine 183 may allow an end user to modify temperature/thermal dose thresholds (as they are further described herein) before, during, or after a given procedure. In yet another example, the UI engine 183 may allow an end user to input information regarding target tissue and non-target tissue, including recommended thresholds, characteristics of types/subtypes of tissue (e.g., temperatures and thermal doses at which damage begins to occur to such tissue types), the type of responses to be performed when a given threshold has been met, and so forth.

As illustrated in FIG. 3, the tissue damage analysis computer system 180 may include the structures and rules engine 184. The structures and rules engine 184 may store and analyze a number of tissue structures and rules related to such tissue structures. For instance, the structures and rules engine 184 may be able to identify and analyze various types of tissue (e.g., brain tissue, skin tissue, kidney tissue, liver tissue, and so forth). The structures and rules engine 184 may further be able to identify and analyze various subtypes of tissue. More specifically, the structures and rules engine 184 may be able to identify and analyze particular types of brain tissue (e.g., the brain stem), skin tissue, kidney tissue, liver tissue, and so forth.

As part of such analyses, the structures and rules engine 184 may (in conjunction with the temperature analysis engine 181 and/or the duration analysis engine 182) be able to determine the maximum temperature and maximum thermal dose that any given tissue type or subtype can withstand before damage to such tissue begins to occur. Based at least partially on these analyses, the tissue damage analysis computer system 180 (and/or one of its engines) may allow for performing a number of functions. For instance, thresholds related to the maximum temperature and/or maximum thermal dose of any given tissue type or subtype may be created for purposes of providing notifications or alerts to an end user (e.g., a physician monitoring a laser ablation procedure), reducing the amount of heat provided by the laser energy source 150, or automatically shutting off one or more devices during a procedure (e.g., the laser energy source 150 during a laser ablation procedure). For instance, when a non-target object of tissue (i.e., a portion of tissue not intentionally being necrotized) that is being monitored by the temperature analysis engine 181 and/or the duration analysis engine 182 reaches a temperature or thermal dose at which necrotization may occur (i.e., a temperature or thermal dose threshold associated with the non-target tissue), the tissue damage analysis computer system 180 may cause one or more devices or pieces of equipment (e.g., the laser energy source 150) to automatically shut off or reduce the laser energy output level.

Such thresholds may be created in a number of different forms. For instance, thresholds for any given tissue type or subtype may be created based on a percent likelihood of damage or on a criticality of the tissue type or subtype. For instance, a threshold for the brain stem may automatically be created to be very conservative (e.g., between 25 and 35 degrees below the temperature at which tissue disruption occurs) because of the importance of the tissue. As such, thresholds may also comprise qualitative identifiers such as conservative, most conservative, least conservative, aggressive, and so forth. In particular, more conservative thresholds may comprise temperatures/thermal doses that are well below the temperatures/thermal doses at which corresponding tissue objects will necrotize. On the other hand, less conservative or aggressive thresholds may comprise temperatures/thermal doses that are at or near the temperatures/thermal doses at which corresponding tissue objects will necrotize or become disrupted.

Notably, such thresholds may automatically be created by the tissue damage analysis computer system 180 based on an analysis of one or more factors, including but not limited to the importance of fully disrupting the target tissue, the criticality of potentially affected non-target tissue, the temperature and/or thermal dose at which the target tissue will necrotize, the temperature and/or thermal dose at which potentially affected non-target tissue will necrotize, the type of procedure being performed, and so forth. For instance, the structures and rules engine 184 may automatically analyze and identify the target tissue and potentially affected objects of non-target tissue based on a type of procedure being performed. Based on such analyses, the structures and rules engine 184 may further consider the importance of necrotizing the target tissue, the criticality of the potentially affected tissue, and the temperature/thermal dose at which necrotization will occur to the potentially affected tissue in comparison to the target tissue. Such analyses and considerations may then be used to generate a threshold temperature and/or thermal dose when a response is to be performed (e.g., sending an alert/notification, lowering heat, shutting off devices, and so forth). As an example, based on determining that potentially affected non-target tissue is critically important, a very conservative threshold may be generated. In another example, a least conservative (or aggressive) threshold may be generated in response to identifying that the target tissue of a given procedure is an advanced malignant tumor.

End users may also be able to generate, modify, or even eliminate temperature/thermal dose thresholds. For instance, the UI engine 183 may allow a user to modify thresholds for any given object of tissue being monitored. In addition, an end user may be able to provide information that can assist the structures and rules engine 184 in generating a threshold for any given tissue. As an example, an end user may be able to identify known objects of tissue near target tissue for a given procedure that may be unintentionally affected by heat applied to the target tissue. Based at least partially on such identification, the structures and rules engine 184 may generate an appropriate threshold for each identified object of tissue. In some embodiments, the UI engine 183 may request that an end user provide any known potentially affected critical tissue for a procedure to be performed.

As described above, there may be situations where equipment is shut off during a procedure (i.e., before the procedure is finished) to protect one or more objects of tissue. In such situations, it may be necessary for the physician performing the procedure to eventually resume and finish the procedure. In such instances, the duration analysis engine 182 may be configured to consider the thermal dose absorbed by all tissue objects affected during all portions of the procedure, regardless of the number of times the procedure is stopped and started again. As an example, during a laser ablation procedure, assume the laser energy source 150 has automatically shut off based on determining that the temperature or thermal dose of a non-target object of tissue has reached its corresponding generated threshold. Also assume that the target tissue must be necrotized, which has not yet occurred. When resuming the procedure and until the procedure is complete, the duration analysis engine 182 may consider the thermal dose absorbed by the non-target tissue during the initial portion of the procedure (i.e., the portion of the procedure prior to automatically shutting off the laser energy source 150) when determining the total thermal dose absorbed by the non-target object of tissue.

Notably, the end user may also be able to modify the threshold corresponding to any given object of tissue to ensure that the procedure can achieve its intended goal. In an example, if the generated threshold for a given non-target object of tissue is initially very conservative and the heat energy source is shut off well before necrotizing a target object of tissue, the end user may be able to adjust the threshold. For instance, a physician may adjust the threshold in situations where disrupting the target tissue is determined to be more important to the patient's well-being than protecting the affected non-target tissue.

FIG. 5 illustrates a flowchart of a method 200 for performing an automated laser ablation procedure. The method 200 includes inputting of user input values (step 210) by a physician or other users into an automated laser ablation system using a GUI. The user input values may include location and size of ablation area. The method 200 further includes receiving of the user input values at a controller (step 220). In some embodiments, the controller may transmit the user input values via a communications interface to a UI engine of a tissue damage analysis computer system. The tissue damage analysis computer system may utilize the user input values to automatically determine parameters of the laser ablation procedure. The parameters may include target tissue type, non-target tissue type, necrotizing tissue temperature or dose of the target tissue and non-target tissue.

The method 200 may further include activating the automated laser ablation system (step 230). Once activated, an MRI engine of the controller directs the MRI device to locate a laser applicator disposed within or adjacent to the target tissue. In certain instances, locating of the laser applicator may require orienting the MRI device (step 240) such that the laser applicator is within a field of capture. Additionally, the MRI engine may direct the MRI device to determine a temperature, using MR thermometry (step 250), of the target tissue and/or the non-target tissue at the ablation site. In some embodiments, the controller may monitor the MR thermometry tissue temperature throughout the ablation procedure. The tissue temperature may be provided to the tissue damage analysis computer system to automatically monitor the tissues temperatures and compare them to limits and thresholds.

The method 200 may further include automatically controlling, via the controller, certain functions of one or more of a laser energy source, a laser fiber manipulating device, and a laser fiber cooling device (step 260). For example, a laser energy source engine of the controller may control functions of the laser energy source that include a power level, a wavelength, and a pulse frequency. Other functions are contemplated. A laser fiber manipulating device engine of the controller may control functions of the laser fiber manipulating device that include direction of axial displacement, distance of axially displacement, speed of axial displacement, timing of axial displacement, degree of radial directionality of a distal end of a laser fiber, and degrees of rotation about a longitudinal axis of the laser fiber. Other functions are contemplated. A laser fiber cooling device engine of the controller may control functions of the laser fiber cooling device that include fluid flowrate, fluid pressure, and fluid temperature. Other functions are contemplated.

The principles described herein may allow for automatically performing a laser ablation during a procedure that utilizes heat to disrupt target tissue by evaluating both the temperature of non-target tissue and the thermal dose absorbed by non-target tissue. In this way, critical tissue structures (e.g., the brain stem) near target tissue (e.g., a malignant brain tumor) may be protected even when disrupting nearby tissue. Notably, without the thermal dose being evaluated, critical tissue at even low levels of elevated temperature may be damaged or disrupted when heat has been applied for relatively long periods of this time. As such, the principles described herein provide much more robust protection for critical tissue structures during a procedure that applies heat to tissue than methods and systems that merely monitor for temperature.

Although the subject matter has been described in language specific to structural features and/or methodological acts, it is to be understood that the subject matter defined in the appended claims is not necessarily limited to the described features or acts described above, or the order of the acts described above. Rather, the described features and acts are disclosed as example forms of implementing the claims.

Any of the steps, functions, and operations discussed herein can be performed continuously and automatically.

While the flowcharts have been discussed and illustrated in relation to a particular sequence of events, it should be appreciated that changes, additions, and omissions to this sequence can occur without materially affecting the operation of the disclosed embodiments, configuration, and aspects.

The exemplary systems and methods of this disclosure have been described in relation to tissue ablation systems and methods. However, to avoid unnecessarily obscuring the present disclosure, the preceding description omits a number of known structures and devices. This omission is not to be construed as a limitation of the scope of the claimed disclosure. Specific details are set forth to provide an understanding of the present disclosure. It should, however, be appreciated that the present disclosure may be practiced in a variety of ways beyond the specific detail set forth herein.

A number of variations and modifications of the disclosure can be used. It would be possible to provide for some features of the disclosure without providing others.

Any methods disclosed herein comprise one or more steps or actions for performing the described method. The method steps and/or actions may be interchanged with one another. In other words, unless a specific order of steps or actions is required for proper operation of the embodiment, the order and/or use of specific steps and/or actions may be modified. For example, a method of controlling a thermal energy or ablation system may include one or more of the following steps: inputting user input values into a graphical user interface; receiving the user input values at an controller coupled to a thermal energy source, a thermal energy fiber positioning device, and a magnetic resonance imaging system; activating the thermal energy or ablation system via the controller; monitoring an MR thermometry tissue temperature at an ablation site; and automatically controlling, via the controller, the thermal energy source, the thermal energy fiber positioning device, and the MRI system during a thermal tissue ablation procedure based on the MR thermometry tissue temperature at the ablation site. Other steps are also contemplated.

Embodiments may be understood by reference to the drawings, wherein like parts are designated by like numerals throughout. It will be readily understood by one of ordinary skill in the art having the benefit of this disclosure that the components of the embodiments, as generally described and illustrated in the figures herein, could be arranged and designed in a wide variety of different configurations. Thus, the following more detailed description of various embodiments, as represented in the figures, is not intended to limit the scope of the disclosure, but is merely representative of various embodiments. While the various aspects of the embodiments are presented in drawings, the drawings are not necessarily drawn to scale unless specifically indicated.

The phrase “coupled to” refers to any form of interaction between two or more entities, including mechanical, electrical, magnetic, electromagnetic, fluid, and thermal interaction. Two components may be coupled to each other even though they are not in direct contact with each other. For example, two components may be coupled to each other through an intermediate component.

“Fluid” is used in its broadest sense, to refer to any fluid, including both liquids and gases as well as solutions, compounds, suspensions, etc., which generally behave as fluids.

References to approximations are made throughout this specification, such as by use of the term “about.” For each such reference, it is to be understood that, in some embodiments, the value, feature, or characteristic may be specified without approximation. For example, where the qualifier “about” is used, the term includes within its scope the qualified word in the absence of their qualifier.

References in the specification to “one embodiment,” “an embodiment,” “an example embodiment,” “some embodiments,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in conjunction with one embodiment, it is submitted that the description of such feature, structure, or characteristic may apply to any other embodiment unless so stated and/or except as will be readily apparent to one skilled in the art from the description. The present disclosure, in various embodiments, configurations, and aspects, includes components, methods, processes, systems and/or apparatus substantially as depicted and described herein, including various embodiments, subcombinations, and subsets thereof. Those of skill in the art will understand how to make and use the systems and methods disclosed herein after understanding the present disclosure. The present disclosure, in various embodiments, configurations, and aspects, includes providing devices and processes in the absence of items not depicted and/or described herein or in various embodiments, configurations, or aspects hereof, including in the absence of such items as may have been used in previous devices or processes, e.g., for improving performance, achieving ease, and/or reducing cost of implementation.

The foregoing discussion of the disclosure has been presented for purposes of illustration and description. The foregoing is not intended to limit the disclosure to the form or forms disclosed herein. In the foregoing Detailed Description for example, various features of the disclosure are grouped together in one or more embodiments, configurations, or aspects for the purpose of streamlining the disclosure. The features of the embodiments, configurations, or aspects of the disclosure may be combined in alternate embodiments, configurations, or aspects other than those discussed above. This method of disclosure is not to be interpreted as reflecting an intention that the claimed disclosure requires more features than are expressly recited in each claim. Rather, as the following claims reflect, inventive aspects lie in less than all features of a single foregoing disclosed embodiment, configuration, or aspect. Thus, the following claims are hereby incorporated into this Detailed Description, with each claim standing on its own as a separate preferred embodiment of the disclosure.

Moreover, though the description of the disclosure has included description of one or more embodiments, configurations, or aspects and certain variations and modifications, other variations, combinations, and modifications are within the scope of the disclosure, e.g., as may be within the skill and knowledge of those in the art, after understanding the present disclosure. It is intended to obtain rights, which include alternative embodiments, configurations, or aspects to the extent permitted, including alternate, interchangeable and/or equivalent structures, functions, ranges, or steps to those claimed, whether or not such alternate, interchangeable and/or equivalent structures, functions, ranges, or steps are disclosed herein, and without intending to publicly dedicate any patentable subject matter.

As used herein, the singular forms “a,” “an,” and “the” are intended to include the plural forms as well, unless the context clearly indicates otherwise. It will be further understood that the terms “include,” “including,” “includes,” “comprise,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof. The term “and/or” includes any and all combinations of one or more of the associated listed items.

The term “automatic” and variations thereof, as used herein, refers to any process or operation, which is typically continuous or semi-continuous, done without material human input when the process or operation is performed. However, a process or operation can be automatic, even though performance of the process or operation uses material or immaterial human input, if the input is received before performance of the process or operation. Human input is deemed to be material if such input influences how the process or operation will be performed. Human input that consents to the performance of the process or operation is not deemed to be “material.”

The terms “determine,” “calculate,” “compute,” and variations thereof, as used herein, are used interchangeably and include any type of methodology, process, mathematical operation, or technique.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and this disclosure.

It should be understood that every maximum numerical limitation given throughout this disclosure is deemed to include each and every lower numerical limitation as an alternative, as if such lower numerical limitations were expressly written herein. Every minimum numerical limitation given throughout this disclosure is deemed to include each and every higher numerical limitation as an alternative, as if such higher numerical limitations were expressly written herein. Every numerical range given throughout this disclosure is deemed to include each and every narrower numerical range that falls within such broader numerical range, as if such narrower numerical ranges were all expressly written herein.

What is claimed is:

1. A thermal ablation control system, comprising:
a memory to store user input values defining elements of a thermal ablation procedure; and
a controller comprising:
a communication interface coupled to a thermal energy source, a thermal fiber positioning device, and a magnetic resonance imaging (MRI) system; and
a processor to:
monitor a magnetic resonance (MR) thermometry tissue temperature at an ablation site; and
automatically control functions of the thermal energy source, the thermal fiber positioning device, and the MRI system via the communication interface during thermal ablation of a target tissue based on the MR thermometry tissue temperature at the ablation site and the user input values stored in the memory.

2. The thermal ablation control system of claim 1, wherein the thermal energy source is configured to output a laser energy having a wavelength, a power level, and a pulse frequency, and wherein the controller is configured to automatically control the wavelength, the power level, or the pulse frequency of the thermal energy source.

3. The thermal ablation control system of claim 1, wherein the thermal fiber positioning device comprises:
an axial positioning mechanism; and
a directionality mechanism, wherein the controller is configured to automatically control the axial positioning mechanism and the directionality mechanism.

4. The thermal ablation control system of claim 1, wherein the MM system comprises an MR thermometry function configured to non-invasively estimate a tissue temperature, and a thermal fiber locating function configured to orient the MM system to locate a thermal fiber relative to the ablation site.

5. The thermal ablation control system of claim 4, wherein the controller is configured to automatically control the MR thermometry function and the thermal fiber locating function.

6. The thermal ablation control system of claim 1, wherein the communication interface is further coupled to a thermal fiber cooling system, wherein the controller is configured to automatically control functions of the thermal fiber cooling system via the communication interface during thermal ablation of the target tissue, wherein the thermal fiber cooling system comprises a fluid pump, and wherein the controller is configured to control a flowrate of the fluid pump.

7. The thermal ablation control system of claim 1, wherein the controller is configured to receive the user input values stored in the memory, and wherein the user input values comprise:
data this is indicative of a protected tissue area; and
data this is indicative of a target tissue area.

8. A method of controlling a thermal ablation system, comprising:
receiving user input values at a controller coupled to a thermal energy source, a thermal fiber positioning device, and a magnetic resonance imaging (MRI) system;
activating the thermal ablation system via the controller;
monitoring a magnetic resonance (MR) thermometry tissue temperature at an ablation site; and
automatically controlling, via the controller, the thermal energy source, the thermal fiber positioning device, and the MM system during a thermal tissue ablation procedure based on the MR thermometry tissue temperature at the ablation site.

9. The method of claim 8, wherein automatically controlling the thermal energy source comprises controlling a wavelength, a power level, or a pulse frequency of a laser energy output of the thermal energy source.

10. The method of claim 8, wherein automatically controlling the thermal fiber positioning device comprises:
controlling an axial positioning mechanism; and
controlling a directionality mechanism.

11. The method of claim 8, wherein automatically controlling the MRI system comprises controlling an MR thermometry function configured to non-invasively estimate a tissue temperature and a thermal fiber location function configured to orient the MM system to locate a thermal fiber relative to the ablation site.

12. The method of claim 8, further comprising automatically controlling a flowrate of a thermal fiber cooling device, the thermal fiber cooling device comprising a fluid pump.

13. A thermal ablation system, comprising:
a thermal energy source;
a thermal fiber positioning device;
a magnetic resonance imaging (MRI) system;
a thermal fiber cooling device; and
a controller comprising a communication interface coupled to the thermal energy source, the thermal fiber positioning device, the MRI system, and the thermal fiber cooling device;
wherein the controller is configured to automatically control functions of the thermal energy source, the thermal fiber positioning device, the MRI system, and the thermal fiber cooling device via the communication interface during thermal ablation of a target tissue.

14. The thermal ablation system of claim 13, wherein the thermal energy source is configured to output a laser energy having a wavelength, a power level, and a pulse frequency.

15. The thermal ablation system of claim 14, wherein the controller is configured to automatically control the wavelength, power level, and pulse frequency of the laser energy output of the thermal energy source.

16. The thermal ablation system of claim 14, wherein the thermal fiber positioning device comprises:
an axial positioning mechanism; and
a directionality mechanism, wherein the controller is configured to automatically control the axial positioning mechanism and the directionality mechanism.

17. The thermal ablation system of claim 13, wherein the MM system comprises a magnetic resonance (MR) thermometry function and a thermal fiber locating function, and wherein the controller is configured to automatically control the MR thermometry function and the thermal fiber locating function.

18. The thermal ablation system of claim 13, wherein the thermal fiber cooling device comprises a fluid pump, and wherein the controller is configured to control a flowrate of the fluid pump.

19. The thermal ablation system of claim 13, wherein the controller is configured to receive user input values comprising:
a protected tissue area; and
a target tissue area.

20. The thermal ablation system of claim 13, wherein the controller is configured to receive a pre-calculated tissue damage model.
